(19) United States
(12) Patent Application Publication
(10) Pub. No.: US 2011/0172163 A1
(43) Pub. Date: Jul. 14, 2011

(54) Novel Protease Inhibitors

(75) Inventors: Jonathan M. Harris, Queensland (AU); Joakim E. Swedberg, Queensland (AU)

(73) Assignee: QUEENSLAND UNIVERSITY OF TECHNOLOGY, Brisbane (AU)

(21) Appl. No.: 13/058,325

(22) PCT Filed: Aug. 11, 2009
(86) PCT No.: PCT/AU09/01031

§ 371 Date: Mar. 29, 2011

(30) Foreign Application Priority Data
Aug. 12, 2008 (AU) 2008904159
Jun. 25, 2009 (AU) 2009902963

(51) Int. Cl. A61K 38/12; C07K 7/00; A61P 35/00; G01N 33/48

(52) U.S. Cl. 514/19.4; 530/317; 514/19.9; 514/19.5; 514/20.3; 436/64

(57) ABSTRACT

The invention relates to KLK protease inhibitors. In particular, the invention is directed to KLK4 protease inhibitors and their uses in the treatment of a cancer, such as prostate cancer.

### TECHNICAL FIELD

The invention described herein relates generally to protease inhibitors. In particular, the invention is directed to kallikrein related peptidase inhibitors and their use in the diagnosis, prevention and treatment of cancer, although the scope of the invention is not necessarily limited thereto.

### BACKGROUND ART

Prostate cancer is presently the most common cancer and leading cause of death among male cancer patients (Center for Health Statistics, 2002; Australian Institute of Health and Welfare, 2001). Current treatments for prostate cancer focus on androgen deprivation and are associated with a plethora of side effects (1) and no effective treatment is currently available for late stages of the disease. Kallikrein related peptidase 3 (KLK3; PSA; hK3) is currently the dominant biomarker for diagnosis and prognosis of prostate cancer. KLK3 belongs to the kallikrein related peptidase (KLK) gene family encoding homologous serine endopeptidases with trypsin or chymotrypsin-like substrate specificity.

Expression of the KLK proteases is modulated by a number of signaling pathways, with the best characterized regulators being sex-steroid hormones (2); these play a vital role in regulation of gene expression both during normal development and in the pathogenesis of hormone dependent cancers. Interestingly, the KLK genes are often expressed as groups within different tissues, and their expression is commonly dysregulated in cancer cells including ovarian (3-5), testicular (3), breast (3,6) and prostate tumours (3,7). As a result, a large number of recent studies have focused on revealing differences in expression patterns of the KLK's between normal and diseased cells, suggesting a role for these enzymes in disease pathogenesis. However, the substrate specificity for many KLK proteases has yet to be elucidated and their physiological substrates are largely unknown.

Kallikrein related peptidase 4 (KLK4) is predominantly expressed in basal and secretory cells of the prostate gland, although lower levels of expression have been detected in a number of tissues including breast, ovaries, thyroid, testis and teeth (5,8-11). Over-expression has been documented in malignant prostate, ovarian and breast tumours (5,11-15). It has also been demonstrated that overexpression of KLK4 in prostate cancer cells is associated with loss of E-cadherin and an increase in vimentin expression, causing an Epithelial to Mesenchymal Transition (EMT)-like effect and increased in vitro cell migration rates (14). Consistent with this, KLK4 is also expressed in malignant breast mesothelioma (13). Furthermore, immunohistochemical analysis of prostate cancer bone metastasis tissue indicates that KLK4 is localized to both prostate cancer cells and oesteoblasts with upregulation of KLK4 expression occurring in prostate cancer cells (LNCaP and PC3) co-cultured with osteoblast-like cells (SaOs2; derived from oestoblastic bone metastasis) (16).

Although current knowledge of the physiological function of KLK4 is incomplete, recent in vitro experiments have suggested several potential substrates and functions that may influence cancer progression at the primary site. These include the proteolysis of fibrinogen and collagen I and IV (17), prostatic acid phosphatase (18, 19), insulin growth factor binding protein (IGFBP-3,-4,-5,-6;) (20), amelogenin (21, 22), the sex hormone-binding globulin (SHBG) (23), urokinase plasminogen activator receptor (uPAR) (24) and activation of the zymogens PSA (Pro-PSA) and single chain urokinase plasminogen activator (Pro-uPA). KLK4′s role during mineralization of tooth enamel is better understood as a KLK4 gene mutation resulting in a truncated inactive protein results in autosomally recessive hypomaturation amelogenesis imperfecta (25). Enamel crystal formation during tooth development occurs by successive cleavage of amelogenin and other secreted enamel matrix proteins; first, during the secretary phase by enamelysin (MMP-20) and later during the maturation phase, by KLK4 (26, 27).

Considering that KLK4 has a role in processing extracellular matrix (ECM) proteins in mineralized tissues and is overexpressed in prostate cancer cells, it has been suggested that KLK4 may facilitate bone metastasis by degradation of bone ECM components (16). The possible dual role of KLK4 in prostate cancer progression and bone metastasis highlights the enzyme's potential as a point of therapeutic intervention. As yet few studies have addressed the lack of small molecule inhibitors for KLK4.

Sunflower trypsin inhibitor (SFTI or SFTI-1) belongs to the Bowmann-Birk (BBI) serine protease inhibitor family. It was recently discovered in the seeds of sunflower (Helianthus annuus) and characterized by determination of its three-dimensional structure in complex with bovine β-trypsin (28). With only 14 amino acid residues and a molecular mass of 1,513 Da, SFTI is the smallest, as well as the most potent (β-trypsin: Kᵢ=0.1 nM) naturally occurring serine protease inhibitor known (28). SFTI is stabilized by both backbone cyclization and a disulfide bridge. Comparison of the structure of SFTI in complex with β-trypsin and in solution (29) indicates that this peptide's conformation does not change markedly upon binding to the protease.

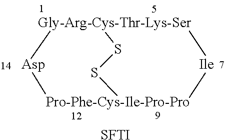

There would be an advantage to provide a small molecule, selective and potent inhibitor of KLK, which may overcome some of the above-mentioned disadvantages or provide a useful or commercial choice.

### SUMMARY OF THE INVENTION

According to a first aspect, the invention provides a compound or salt thereof according to Formula I:

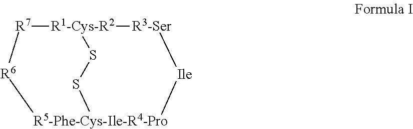

wherein

R¹, R², R³, R⁴, R⁵, R⁶ and R⁷ are each an amino acid residue; and

wherein when R⁴ is proline, R² is an amino acid residue other than threonine.

In one embodiment, R¹ is Phe, R² is Gln and R³ is Arg.

In another embodiment, R¹ is Phe, R² is Gln, R³ is Arg, and R⁶ is Asn.

According to a second aspect of the invention, there is provided a pharmaceutical or veterinary composition for the diagnosis, prevention or treatment of a cancer in a mammalian subject, the composition including at least one compound according to the first aspect together with a pharmaceutically or veterinarially acceptable carrier or diluent.

In one embodiment, the cancer is prostate cancer.

According to a third aspect of the invention, there is provided a compound according to the first aspect for the diagnosis, prevention or treatment of a cancer in a mammalian subject.

In one embodiment, the cancer is prostate cancer.

According to a fourth aspect of the invention, there is provided a method for the diagnosis, prevention or treatment of a cancer in a mammalian subject, the method including administering to the subject a therapeutically effective of at least one compound according to the first aspect.

In one embodiment, the cancer is prostate cancer.

According to a fifth aspect of the invention, there is provided a method for diagnosis, prevention or treatment of a KLK-mediated disease in a mammalian subject, the method including administering to the subject a therapeutically effective amount of at least one compound according to the first aspect.

In one embodiment, the KLK-mediated disease is a KLK4-mediated disease.

In another embodiment, KLK-mediated disease is prostate cancer.

According to a sixth aspect of the invention, there is provided a method of identifying a compound according to the first embodiment comprising:
a. synthesizing tetrapeptides of formula II:

R¹-Cys-R²—R³ Formula II

wherein R¹ and R³ are each an amino acid residue; and R² is an amino acid residue other than threonine;
b. contacting the tetrapeptides with an assay of a protease of interest;
c. selecting tetrapeptides with a relatively rapid rate of cleavage;
d. replacing amino acid residues at positions 2 to 5 of SFTI with the selected tetrapeptides.

With reference to Formulae I and II, R¹, R², R³, R⁴, R⁵, R⁶ and R⁷ can be any amino acid residue, including the residues of natural and unnatural amino acids. R¹, R², R³, R⁴, R⁵, R⁶ and R⁷ can be any conformation, but are preferably α-amino acid residues. R¹ is preferably a non-polar amino acid residue; R² is preferably a neutral amino acid residue, and R³ is preferably a basic amino acid residue.

In order that the invention may be more readily understood and put into practice, one or more preferred embodiments thereof will now be described, by way of example only, with reference to the accompanying drawings.

### BRIEF DESCRIPTION OF THE DRAWINGS

FIG. 1. Amidolytic activity of KLK4 against sparse matrix library pNA substrates. The y-axes represents the rate of substrate cleavage in mOD /min(405), with the amino acid at position P2 indicated beside each y-axis (numbering of the positions on the substrates is according to [40]). The x-axis labels the bars corresponding to substrates with each of the 25 combinations of the amino acids in the P4 and P3 positions. Arginine was kept constant in the P1 position while varying the amino acid in positions P2-P4 as indicated in the graph. Data represented as mean±SEM from three experiments in triplicate.

FIG. 2 Inhibition of serine protease proteolytic activity by SFTI and SFTI-FCQR. Kallikrein and trypsin-mediated proteolysis of fibrinogen assessed by SDS-PAGE. Proteolytic products were visualized by Coomassie blue staining following resolution on 12% polyacrylamide gels. (A) Effect of increasing concentrations of SFTI-FCQR on fibrinogen digestion by KLK4 and SFTIwt at a concentration of 2000 nM. (B) Trypsin digestion of fibrinogen in the presence and absence of 2000 nM SFTI-FCQR. (C-F) Digestion of fibrinogen by KLK2, 5, 12, and 14, respectively, in the presence of increasing concentrations of SFTI-FCQR. Gels are representative of three independent experiments.

FIG. 3. Stability of SFTI variants in contact with prostate cancer cells in vitro. Residual SFTI-FCQR was assayed in tissue culture supernatants from prostate cancer cells treated with a single dose of inhibitor at time =0. Stability was assessed against LNCaP (closed circles), 22RV1 (open circles) and PC3 cells (triangles) for (A) SFTI-FCQR, (B) SFTI-FCQR Asn14 and (C) SFTI-FCQR Lys14 as summarized in (D). Data represented as mean±SEM from three independent experiments in triplicates.

FIG. 4. Effect of SFTI-FCQR on calcium release in cell based assays. Lung murine fibroblasts (LMF) stably expressing human PAR-2 were incubated after 30 seconds with either activated iKLK4 (300 nM), trypsin (10 nM), PAR-2 Activating peptide (100 nM) with or without SFTI-FCQR (1 μM). The fluorescence at 510 nm was measured following alternating excitation at 340 and 380 nm (Em510(340/380)). The ratio of Em510(340/380) is proportional to intracellular Ca²⁺ ion concentration. The data is represented as mean of three experiments in triplicate. Treatment with (A) Buffer or (B) SFTI-FCQR had no effect while treatment with (C) trypsin, (D) iKLK4, (E) trypsin and SFTI-FCQR, or (F) PAR-2 Activating peptide resulted in increased intracellular Ca²⁺ ion concentration. Calcium flux was restored in cells initially treated with iKLK4 and SFTI-FCQR at 30 sec by addition of (G) trypsin or (H) PAR-2 activating peptide after 3.5 minutes. Arrows indicate injection points for solutions as indicated.

FIG. 5. Effect of KLK4 inhibitor on primary tumour volume of LNCaP cells in an animal model of prostate cancer. Nude mice were injected with 1.5×10⁶ LNCaP prostate cancer cells stably expressing an exogenous luciferase reporter gene and imaged over a period of 56 days. Tumour growth was measured by injecting mice with luminol and the resulting light emissions were quantified using a xenogen in vivo imaging system. When tumour specific growth rate was less than 2%/day, administration of 3×i.v. doses of 0.2 mg/kg SFTI-FCQR (open) arrested tumour growth rate in comparison to control (closed).

FIG. 6. IC₅₀ Values for SFTI and SFTI variants with various proteases. (A) Determination of IC₅₀ of KLK4 with the WT-SFTI (open circles; 221.4±1.10 nM) and that of SFTI-FCQR IC₅₀ with KLK4 (closed circles; 7.97±1.08 nM), KLK5 (squares; 2348±721 nM) and KLK14 (triangles; 1506±37.1 nM. (B) Determination of IC₅₀ values for SFTI-FCQR Asn14 with KLK4 (closed circles; 0.063±0.002 nM), KLK14 (closed triangles; 250.8±6.65 nM) and trypsin (open circles 2579±507.7 nM), as well as SFTI-FCQR Lys14 with KLK4 (squares; 98.94±4.09 nM) and SFTI-FCQR Ala9 with KLK4 (open triangles; 1142±76 nM). (C) Comparison of inhibition of KLK4 by the SFTI variants SFTI-FCQR Asn14 (closed circles; 0.063±0.002 nM), SFTI-FCQR (open triangles; 7.97±1.08 nM), SFTI-FCQR Lys14 (closed triangles; 98.94±4.09 nM), and SFTI-FCQR Ala9 (squares; 1142±76 nM). Data expressed as mean±SEM from three independent experiments in triplicates.

FIG. 7. SDS-PAGE analysis of inhibition of trypsin and KLK12 fibrinogen digestion by SFTI variants. The WT-SFTI completely inhibits trypsin digestion of fibrinogen at a concentration of 31.25 nM (A), while neither the SFTI-FCQR Lys14 (B) nor the SFTI-FCQR Asn14 variant (C) inhibited trypsin digestion of fibrinogen at a concentration of 10000 nM. Similarly, the SFTI-FCQR Asn14 variant did not inhibit KLK12 (D) or KLK14 (E) digestion of fibrinogen at a concentration of 10000 nM. Gels are representative of three independent experiments.

### DETAILED DESCRIPTION OF PREFERRED EMBODIMENTS

The following explanation of terms, abbreviations and methods are provided to better describe the present compounds, compositions and methods. It is understood that the terminology used in the disclosure is for the purpose of describing particular embodiments and examples only, and is not intended to be limiting.

### ABBREVIATIONS

SFTI: Sunflower trypsin inhibitor
SFTI-FCQR: SFTI variant with 2-Phe, 3-Cys, 4-Gln and 5-Arg
SFTI-FCQR Ala9: SFTI variant with 2-Phe, 3-Cys, 4-Gln, 5-Arg and 9-Ala
SFTI-FCQR Lys14: SFTI variant with 2-Phe, 3-Cys, 4-Gln, 5-Arg and 14-Lys
SFTI-FCQR Asn14: SFTI variant with 2-Phe, 3-Cys, 4-Gln, 5-Arg and 14-Asn
IPTG: Isopropyl-β-D-thiogalactoside;
DMF: N,N-dimethylformamide;
DBU: 1,8-Diazabicyclo [5.4.0]undec-7-ene;
HBTU: 2-(1H-benzotriazole-1-yl)-1,1,3,3-tetramethyluronium hexafluoro-phosphate;
HOBT: 1-Hydroxybenzotriazole;
DIPEA: N,N-Diisopropylethylamine;
DTT: Dithothreitol;
FMOC: 9-fluorenylmethyl carbamate;
SDS: Sodium dodecyl sulfate;
PAGE: Polyacrylamide gel electrophoresis;
PAR Protease activated receptor
KLK4: Kallikrein related peptidase 4;
KLK3: Kallikrein related peptidase 3;
KLK: Kallikrein related peptidases;
TFA: Trifluoroacetic acid;
DCM: Dichlormethane:
MS: Mass spectroscopy;
TOF: Time of flight;
MALDI: Matrix assisted laser desorption ionization;
pNA: para-Nitroanilide;
EIS: Electron ionization spray;
Bz: Benzyl;
Ac: Acetyl;
IGFBP: insulin growth factor binding protein;
SHBG: sex hormone-binding globulin;
uPAR: urokinase plasminogen activator receptor;
Pro-uPA: pro-urokinase plasminogen activator;
MMP-20: Enamelysin.

The term “amino acid residue” refers to both natural and unnatural amino acid residues in their D and L steroisomers for chiral amino acid residues. Moreover, peptide compounds disclosed herein may contain asymmetric centers in addition to the chiral centers in the backbone of the peptide compound. These asymmetric centers may independently be in either the R or S configuration. It will also be apparent to those skilled in the art that certain peptide compounds disclosed herein may exhibit geometrical isomerism. Geometrical isomers include the cis and trans forms of peptide compounds of the invention having alkenyl moieties. The present compounds comprise the individual geometrical isomers and stereoisomers and mixture thereof

Natural and unnatural amino acids are well known to those of ordinary skill in the art. Common natural amino acids include, without limitation, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Uncommon and unnatural amino acids include, without limitation, allyl glycine (AllylGly), biphenylalanine (Bip), citrulline (Cit), 4-guanidinophenylalanine (Phe(Gu)), homoarginine (hArg), homolysine (hLys), 2-napthylalanine (2-Nal), ornithine (Orn) and pentafluorophenylalanine (F₅Phe).

Amino acids are typically classified in one or more categories, including polar, hydrophobic, acidic, basic and aromatic, according to their side chains. Examples of polar amino acids include those having side chain functional groups such as hydroxyl, sulfhydryl, and amide, as well as the acidic and basic amino acids. Polar amino acids include, without limitation, asparagine, cysteine, glutamine, histidine, selenocysteine, serine, threonine, tryptophan and tyrosine. Examples of hydrophobic or non-polar amino acids include those residues having nonpolar aliphatic side chains, such as, without limitation, leucine, isoleucine, valine, glycine, alanine, proline, methionine and phenylalanine Examples of basic amino acids include those having a basic side chain, such as an amino or guanidine group. Basic amino acids include, without limitation, arginine, homolysine and lysine. Examples of acidic amino acids include those having an acidic side chain functional group, such as carboxy group. Acidic amino acids include, without limitation aspartic acid and glutamic acid. Aromatic amino acids include those having and aromatic side chain group. Examples of aromatic amino acids include, without limitation, biphenylalanine, histidine, 2-napthylalananine, pentafluorophenylaline, phenylalanine, tryptophan and tyrosine. It is noted that some amino acids are classified in more than one group, for example, histidine, tryptophan and tyrosine and classified as both polar and aromatic amino acids. Additional amino acids that are classified in each of the above groups are known to those of ordinary skill in the art.

The peptide compounds of the disclosure can be prepared using virtually any technique known to one of ordinary skill in the art for the preparation of peptides. For example, the peptide compounds can be prepared using step-wise solution or solid phase peptide syntheses, or recombinant DNA techniques, or the equivalents thereof.

Peptide compounds of the disclosure having either the D- or L-configuration can be readily synthesized by automated solid phase procedures well known in the art. Suitable syntheses can be performed by utilizing “T-boc” or “F-moc” procedures. Techniques and procedures for solid phase synthesis are described in Solid Phase Peptide Synthesis: A Practical Approach, by E. Atherton and R. C. Sheppard, published by IRL, Oxford University Press, 1989. Alternatively, the peptide compounds may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). This is particularly the case with glycine containing peptides. Other methods useful for synthesizing the peptide compounds of the disclosure are described in Nakagawa et al., J. Am. Chem. Soc. 107:7087-7092, 1985.

Additional exemplary techniques known to those of ordinary skill in the art of peptide and peptide analog synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups.

If a peptide compound is composed entirely of gene-encoded amino acids, or a portion of it is so composed, the peptide compound or the relevant portion can also be synthesized using conventional recombinant genetic engineering techniques. For recombinant production, a polynucleotide sequence encoding the peptide compound is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable target cell which will express the peptide compound. Depending on the expression system used, the expressed peptide is then isolated by procedures well-established in the art. Methods for recombinant protein and peptide production are well known in the art (see, e.g., Sambrook et al. (ed.), Molecular Cloning: A Laboratory Manual, 2^{nd} ed., vol. 1-3, Cold Spring Harbor Laboratory Press, Cold Spring Harbor, N.Y., 1989, Ch. 17 and Ausubel et al. Short Protocols in Molecular Biology, 4^{th} ed., John Wiley & Sons, Inc., 1999).

To increase efficiency of production, the polynucleotide can be designed to encode multiple units of the peptide compound separated by enzymatic cleavage sites. The resulting polypeptide can be cleaved (e.g., by treatment with the appropriate enzyme) in order to recover the peptide units. This can increase the yield of peptides driven by a single promoter. In one embodiment, a polycistronic polynucleotide can be designed so that a single mRNA is transcribed which encodes multiple peptides, each coding region operatively linked to a cap-independent translation control sequence, for example, an internal ribosome entry site (IRES). When used in appropriate viral expression systems, the translation of each peptide encoded by the mRNA is directed internally in the transcript, for example, by the IRES. Thus, the polycistronic construct directs the transcription of a single, large polycistronic mRNA which, in turn, directs the translation of multiple, individual peptides. This approach eliminates the production and enzymatic processing of polyproteins and can significantly increase yield of peptide driven by a single promoter.

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedron promoter can be used. When cloning in plant cell systems, promoters derived from the genome of plant cells (e.g., heat shock promoters, the promoter for the small subunit of RUBISCO, the promoter for the chlorophyll a/b binding protein) or from plant viruses (e.g., the 35S RNA promoter of CaMV, the coat protein promoter of TMV) can be used. When cloning in mammalian cell systems, promoters derived from the genome of mammalian cells (e.g., metallothionein promoter) or from mammalian viruses (e.g., the adenovirus late promoter, the vaccinia virus 7.5 K promoter) can be used.

The peptide compounds of the disclosure can be purified by many techniques well known in the art, such as reverse phase chromatography, high performance liquid chromatography, ion exchange chromatography, size exclusion chromatography, affinity chromatography, gel electrophoresis, and the like. The actual conditions used to purify a particular peptide will depend, in part, on synthesis strategy and on factors such as net charge, hydrophobicity, hydrophilicity, and the like, and will be apparent to those of ordinary skill in the art.

A detectable moiety can be linked to the peptide compounds disclosed herein, creating a peptide-detectable moiety conjugate. Detectable moieties suitable for such use include any composition detectable by spectroscopic, photochemical, biochemical, immunochemical, electrical, optical or chemical means. The detectable moieties contemplated for the present disclosure can include, but are not limited to, an immunofluorescent moiety (e.g., fluorescein, rhodamine, Texas red, and the like), a radioactive moiety (e.g., ³H, ³²P, ¹²⁵I, ³⁵S), an enzyme moiety (e.g., horseradish peroxidase, alkaline phosphatase), a colorimetric moiety (e.g., colloidal gold, biotin, colored glass or plastic, and the like). The detectable moiety can be linked to the peptides at either the N- and/or C-terminus. Optionally, a linker can be included between the peptide and the detectable moiety.

Means of detecting such moieties are well known to those of skill in the art. Thus, for example, radiolabels may be detected using photographic film or scintillation counters, while fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The present inventors have found that a broad range of compounds with protease inhibition properties can be identified and synthesised using the strategy illustrated below in the examples. These compounds have utility in the diagnosis, prevention or treatment in mammalian subjects of cancer, and in particular prostate cancer. This utility results from the ability of the compounds to modulate the activity of proteases involved in disease processes. One embodiment of the protease inhibitors disclosed herein includes compounds according to Formula I and their corresponding pharmaceutically acceptable salts.

With reference to Formula I, in one embodiment a cystine bridge, formed by cysteine residues linked through their side chains by a disulfide bond (cysteine-S—S-cysteine), stabilizes the protease inhibitor. In another embodiment, the protease inhibitor is stabilized by a crosslinking group. Examples of crosslinking groups include, but are not limited to, amides, esters, thioesters, ethers, sulfides, disulfides, diselenides, and aromatic and aliphatic groups, such as optionally substituted lower aliphatic carbon chains.

In particular, the invention provides potent KLK4 inhibitors that do not inhibit trypsin or thrombin (involved in the vital blood clothing cascade).

Preferred compounds according to the first embodiment of the invention as defined above include those embraced by generic Formula I and discussed below.

As indicated above, the compounds according to the invention have utility in the diagnosis, prevention or treatment in mammalian subjects of cancer, including, for example, breast cancer, lung cancer, esophageal cancer, prostate cancer, colorectal cancer, ovarian cancer, cervical cancer, testicular cancer, pancreatic cancer, liver cancer, bladder cancer, and kidney cancer. In particular embodiments, the cancer is prostate cancer.

Diagnosis of cancer using the disclosed peptide compounds can be achieved using methods well known in the art, including, for example, detecting the binding of a peptide of the invention with one or more KLK proteases in a sample from a subject. The basic principle of an assay used to identify a KLK protease that binds to a peptide of the invention involves contacting a peptide of the invention and a sample suspected of including one or more KLK proteases from a subject under conditions and for a time sufficient to allow the two components to interact and bind, thus forming a complex which can be detected.

The binding assays can be conducted in a variety of ways. For example, one method to conduct such an assay involves anchoring one or more labeled (either directly or indirectly) peptides of the invention to a solid support (such as, a microarray or in a microtitre plate), contacting the solid support with a sample suspected of including one or more KLK proteases and detecting peptide/protease complexes anchored to the solid support at the end of the reaction. Each of the peptides may be present on the solid support in one or more addressable positions. Alternatively, a sample suspected of including one or more KLK proteases is attached to a solid support and one or more detectable (i.e, labeled) peptides disclosed herein are applied to the solid support, followed by detection of peptide/protease complexes anchored to the solid support at the end of the reaction.

The compounds have particular utility in the treatment of the foregoing disorders in humans. The compounds are typically administered as a component of a pharmaceutical composition as described herein. Pharmaceutical compositions for oral administration can be in tablet, capsule, powder or liquid form. A tablet can include a solid carrier such as gelatine or an adjuvant or an inert diluent. Liquid pharmaceutical compositions generally include a liquid carrier such as water, petroleum, animal or vegetable oils, a mineral oil or a synthetic oil. Physiological saline solution, or glycols such as ethylene glycol, propylene glycol or polyethylene glycol may be included. Such compositions and preparations will generally contain at least 0.1 wt % of the compound.

Parenteral administration includes administration by the following routes: intravenously, cutaneously or subcutaneously, nasally, intramuscularly, intraocularly, transepithelially, intraperitoneally and topically. Topical administration includes dermal, ocular, rectal, nasal, as well as administration by inhalation or by aerosol means. For intravenous, cutaneous or subcutaneous injection, or injection at a site where treatment is desired, the active ingredient will be in the form of a parenterally acceptable aqueous solution which is pyrogen-free and has suitable pH, isotonicity and stability. Those of skill in the art will be well able to prepare suitable solutions using, for example, solutions of the subject compounds or derivatives thereof.

In addition to the at least one compound and a carrier or diluent, compositions according to the invention can further include a pharmaceutically or veterinarially acceptable excipient, buffer, stabiliser, isotonicising agent, preservative or antioxidant or any other material known to those of skill in the art. It will be appreciated by the person of skill that such materials should be non-toxic and should not interfere with the efficacy of the compound(s). The precise nature of any additive may depend on the route of administration of the composition: that is, whether the composition is to be administered orally or parenterally. With regard to buffers, aqueous compositions typically include such substances so as to maintain the composition at a close to physiological pH or at least within a range of about pH 5.0 to about pH 8.0.

Compositions according to the invention can also include active ingredients in addition to the at least one compound. Such ingredients will be principally chosen for their efficacy as anti-cancer agents but can be chosen for their efficacy against any associated condition. Anti-cancer agents include, but are not limited to, therapeutically effective amounts of radiomimetic agents, such as bleomycin and neocarcinostatin; Topoisomerase I inhibitors, such as camptothecins (for example, topotecan and irinotecan), indolocarbazole derivatives, indenoisoquinolines, and homocamptothecins; Topoisomerase II inhibitors, such as epipodophyllotoxins (for example, etoposide and teniposide), anthraclyclines (for example, doxorubicin, idarubicin and epirubicin), ellipticines, and acridines (for example, m-AMSA); and agents that target DNA, such as DNA alkylating agents (cyclophosphamide, chlorambucil, melphalan, BCNU, and platinum derivatives) and ecteinascidin 743. Compositions according to the invention can be administered prior to or subsequent to administration of an anti-cancer agent. Alternatively, the compositions can be administered simultaneously with the administration of an anti-cancer agent.

A pharmaceutical or veterinary composition according to the invention will be administered to a subject in either a prophylactically effective or a therapeutically effective amount as necessary for the particular situation under consideration. The actual amount of at least one compound administered by way of a composition, and rate and time-course of administration, will depend on the nature and severity of the condition being treated or the prophylaxis required. Prescription of treatment such as decisions on dosage and the like will be within the skill of the medical practitioner or veterinarian responsible for the care of the subject. Typically however, compositions for administration to a human subject will include between about 0.01 and 100 mg of the compound per kg of body weight and more preferably between about 0.1 and 10 mg/kg of body weight.

The compounds can be included in compositions as pharmaceutically or veterinarially acceptable derivatives thereof. As used herein “derivatives” of the compounds includes salts, coordination complexes with metal irons such as Mn²⁺ and Zn²⁻, esters such as in vivo hydrolysable esters, free acids or bases, hydrates, or prodrugs. Compounds having acidic groups such as phosphates or sulfates can form salts with alkaline or alkaline earth metals such as Na, K, Mg and Ca, and with organic amines such as triethylamine and Tris (2-hydroxyethyl) amine. Salts can also be formed between compounds with basic groups, such as amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques that will be well known to those of skill in the art.

Prodrug derivatives of the compounds of the invention can be transformed in vivo or in vitro into the parent compounds. Typically, at least one of the biological activities of a parent compound may be suppressed in the prodrug form of the compound, and can be activated by conversion of the prodrug to the parent compound or a metabolite thereof. Prodrugs of compounds of the invention include the use of protecting groups which may be removed in vivo to release the active compound or serve to inhibit clearance of the drug. Suitable protecting groups will be known to those of skill in the art.

Compounds according to the invention have utility in the manufacture of a medicament for the diagnosis, prevention or treatment in mammalian subjects of cancer, and in particular prostate cancer. Processes for the manufacture of such medicaments will be known to those of skill in the art and include the processes used to manufacture the pharmaceutical compositions described above.

Having broadly described the invention, non-limiting examples of the compounds, their synthesis, and their biological activities, will now be given.

### EXAMPLES

### Experimental Procedures

### Sparse Matrix Peptide Library Design and Peptide Synthesis

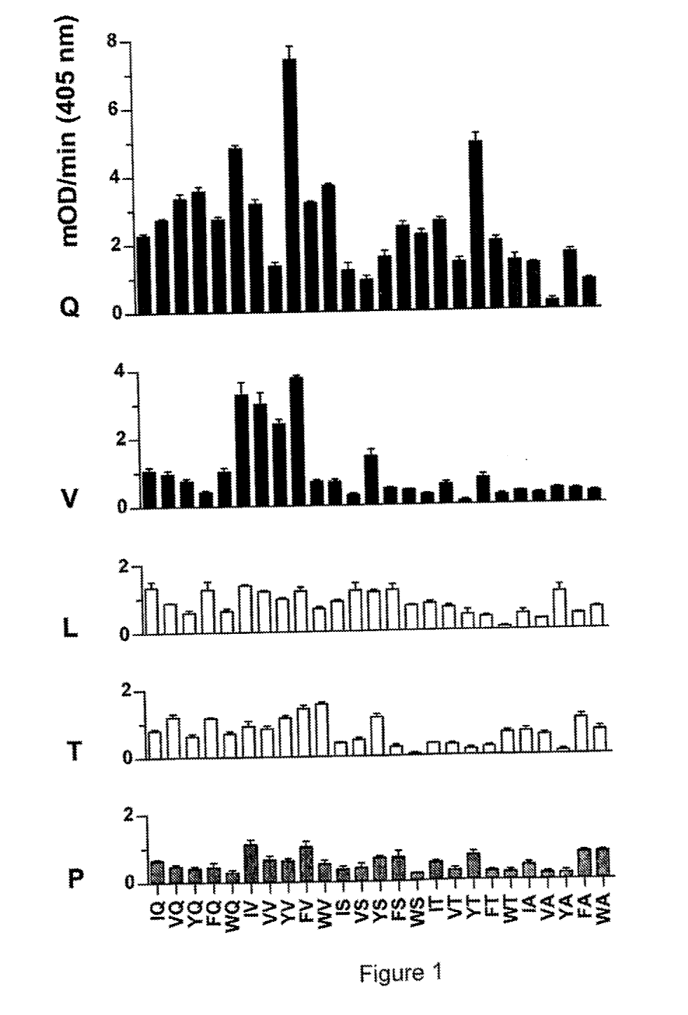

The sparse matrix library was based on Debela [32] and Matsumura [20] PS-SCL screens taking the top 5 scoring sidechains at positions P1-P4, producing the matrix shown in FIG. 1. Analysis of the interaction of SFTI with KLK4 showed that residues beyond Arg2 (P4 position on the inhibitor, sub-site S4 on the enzyme) did not contact the enzyme and hence the library was restricted to tetrapeptides. Peptides were produced using standard solid phase synthesis protocols with 9-fluorenylmethyl carbamate (Fmoc) as semi-permanent protecting group. Peptide elongation was performed with 4 eq. Fmoc amino acids dissolved in 0.25 M each of 2-(1H-benzotriazole-1-yl)-1,1,3,3-tetramethyluronium hexafluorophosphate (HBTU) /1-hydroxybenzotriazole(HOBT)/N,N-Diisopropylethylamine (DIPEA) in N,N-dimethylformamide (DMF). Fmoc deprotection was achieved using 20% piperidine and 5% 1,8-Diazabicyclo [5.4.0]undec-7-ene DBU in DMF.

Peptide-pNAs were synthesized as previously described [37] while peptide aldehydes were synthesised as above using H-Arg(Boc)-H Novasyn TG resin (Novabiochem). After removal of protecting groups with 95% trifluoroacetic acid with scavengers, cleavage was performed using 3×15 ml of acetic acid/H2O/dichloromethan/methanol (10:5:63:21) over 45 minutes.

SFTI was synthesized as a linear molecule on Fmoc-Asp(ODmab)-OH (Bachem) derivitised (0.5 mmol/g) 2-chloro trityl resin (Auspep). Using Fmoc-Cys(STBU)-OH (Bachem) enabled selective removal of the cysteine side chain protecting group (20 eq. of dithiothreitol and 0.5 M DEIPA in DMF) before disulfide bond formation by over night stirring in 10 mM reduced glutathione and 1 mM oxidized glutathione in Tris HCl pH 8.0. Subsequent selective deprotection of the asparagine side chain Dmab protecting group (five washes of two volumes 2% hydrazine in DMF enabled on resin cyclisation through the asparagine side chain using 4 eq. each of DIPEA and 1-[Bis(dimethylamino)methylene]-1H-1,2,3-triazolo[4,5-b]pyridinium 3-oxide hexafluorophosphate (HATU) in DMF. Protecting group removal and cleavage was performed using 95% trifluoroacetic acid with scavengers. Lyophilised synthesis products were purified by reverse phase HPLC from 10% isopropanol, 0.1% TFA in water with a Spherex 5 micron C18 column (Phenomenex), eluting with a linear gradient of 100% isopropanol and 0.1% TFA over 70 minutes. Molecular masses were determined using Protein Chip Send Arrays (Biorad; #C57-30081) with a Protein Chip SELDI-TOF-MS system (Biorad) in accordance with the manufacturer's instruction.

SFTI variants are indicated by the nomenclature SFTI-X¹X²X³X⁴, where X¹-X⁴ are amino acid residues at positions 2-5 of SFTI. For example SFTI-FCQR indicates an SFTI variant with 2-Phe, 3-Cys, 4-Gln and 5-Arg. Variants of the aforementioned are then indicated by the substitution and position of the substitution. For example, SFTI-FCQR Ala9 indicates an SFTI variant with 2-Phe, 3-Cys, 4-Gln, 5-Arg and 9-Ala, while SFTI-FCQR Lys14 indicates an SFTI variant with 2-Phe, 3-Cys, 4-Gln, 5-Arg and 14-Lys and SFTI-FCQR Asn14 indicates an SFTI variant with 2-Phe, 3-Cys, 4-Gln, 5-Arg and 14-Asn.

### Protein Expression and Purification

Human recombinant KLK4 was expressed in E. coli using a previously described chimeric KLK4 plasmid construct where the native pro-region was replaced with the pro-region of PSA, enabling autoactivation [19]. Protein was expressed as inclusion bodies which were isolated and solubilised as described previously to yield 90% pure KLK4. Protein refolding was performed by dilution into 100 volumes of refolding buffer I (2 M urea, 0.1M NaCl, 5 mM GSH, 0.5 mM GSSG, 10 mM benzamidine, 50 mM tris HCl pH 8.0, 2 mM CaCl₂) over 44 hours before further dilution into 3 volumes refolding buffer II (refolding buffer I with 0.5 M urea 0.5 mM GSH, 0.05 mM GSSG, 2 mM CaCl₂) over another 24 hours. Dialysis was performed as previously described [19] (with additional 2 mM CaCl₂ to reduce aggregation [35]) before concentration using UNO sphere Q (Biorad) eluting with tris buffered saline (pH 7.5)/2 mM CaCl₂. Subsequently, the material was purified to apparent homogeneity (according to coomassie blue stained SDS PAGE) by gel filtration using Sephacryl S-200 (GE Health Care) pre-equilibrated with 50 mM Tris-HCl pH 7.5, 20 mM NaCl and 2 mM CaCl₂.

Glycosylated insect-derived KLK4 (iKLK4) was produced in its native form using a previously described construct expression construct where the human KLK4 open reading frame, including the prepro region, is inserted into the pIB/V5-His vector (Invitrogen) before expression in insect Sf9 cells [41]. Purification, dialysis and activation were performed as previously described [42].

### Chromogenic Substrate Screen

Peptide substrates were adjusted to equimolarity according to their absorbance at 405 nm following total hydrolysis of the pNA moiety. Assays were performed in transparent 96 micro-well plates with 4.9 ng of KLK4 and 12 μM substrate in 300 μL assay buffer (0.1 M Tris HCl pH 7.5, 0.1 M NaCl). Hydrolysis was monitored at 405 nm over 14 minutes using a Benchmark plus micro spectrophotometer (Biorad).

Kinetic Studies Active site titration was carried out according to Beynon et al. [38], replacing α₁-proteinase inhibitor with soybean trypsin inhibitor. Enzymatic activity assays were performed with 4.9 nM KLK4 and substrate concentrations in the range of 0-600 μM in 300 μL of assay buffer with hydrolysis monitored over 5 minutes as described for the substrate screen. The data was fitted to the Michaelis-Menten equation by linear regression analysis using the Prism 5 software suite (GraphPad Software Inc.).

### Inhibitor Kinetics

All enzymes and substrates were obtained from Sigma Chemicals if not otherwise specified. Bacterially expressed human KLK4 (37 ng), bovine β-trypsin (20 ng), bovine thrombin (20 ng), bovine α-chymotrypsin (0.5 μg), protease (0.5 μg; Streptomyces griseus type XIV), bovine plasminogen (0.5 μg), and human matriptase (30 ng; R&D Systems) were incubated with various concentrations of inhibitors in 200 μL assay buffer for 10 minutes prior to initiation by the addition of 100 μL of 0.3 mM of peptide-pNA substrates (FVQR-pNA: KLK4; BAPNA: trypsin, (Streptomyces griseus Type XIV Protease and bovine plasma plasminogen); Bz-Phe-Val-Arg-pNA: thrombin and matriptase; Trp-pNA: α-chymotrypsin; Leu-pNA: (Streptomyces griseus Type XIV Protease and bovine plasma plasminogen). The release of pNa moiety was monitored as described above for 7 minutes with each point being the average of three independent triplicate reactions. IC₅₀ was determined using the Prism 5 software suite (GraphPad software Inc.).

### Stability of SFTI-FCQR in Cell Based Systems

Cell monolayers of LNCaP, 22Rv1 and PC3 cells were established in RPMI 1640 medium containing 10% fetal calf serum. Each cell line was treated +/−1 μM SFTI-FCQR contained in 4.0 mL RPMI media (as above) with a vehicle control to account for inhibition by intrinsic media factors. 25 μL samples of media were taken at 0, 24, 48, 72, 96 and 120 hrs timepoints. Media samples were boiled at 97° C. for 15 min, centrifuged at 14,000 rpm for 5 min to remove cellular debris and precipitated protein. Residual SFTI-FCQR inhibitory activity in the medium was determined against recombinant KLK4 using FVQR-pNA as a peptide substrate as above using 15ng KLK4 and 30 nmol FVQR-pNA with 2.5 μL and 50 μL of media+/−SFTI-FCQR added from each treatment.

### Protein Proteolysis

Fibrinogen was solubilised in assay buffer to a final concentration of 1 mg/ml and digested with KLK4 (+/−inhibitor) over a period of 90 minutes at 37° C. Typically assays consisted of 5 μM protein substrate and 2.5 nM KLK4 protein or 1 nM trypsin made up to a final volume of 8 μl with assay buffer. Proteolysis was initiated by addition of KLK4 and terminated by addition of SDS PAGE sample buffer and heat denaturation at 100° C. for 5 minutes. Hydrolysis products were resolved on 10% SDS PAGE gels and compared with incubations terminated immediately after initiation (time 0), and substrate proteins incubated in assay buffer for 90 minutes in the absence of KLK4 or 4-16 mins with trypsin.

### Measurement of Changes in Intracellular Ca²⁺

Lung murine fibroblasts (LMF) from Par-1 null mice stably expressing human PAR-2 were grown to 80% confluence as previously described [41], washed with PBS, detached nonenzymatically, resuspended (4×10⁶ cells/ml) in extracellular medium (121 mM NaCl, 5.4 mM KCl, 0.8 mM MgCl₂, 1.8 mM CaCl₂, 5.5 mM glucose, 25 mM HEPES (pH 7.4)) containing 0.2% (w/v) bovine serum albumin (Sigma) and then loaded with the fluorescence indicator Fura-2 acetoxymethyl ester (1.0 μM; Invitrogen) at room temperature for 60 min. Cells were then pelleted followed by resuspension in extracellular medium (without bovine serum albumin) at a concentration of 2×10⁶ cells/ml for fluorescence measurements. The ratio of fluorescence at 510 nm after excitation at 340 and 380 nm was monitored (Polarstar Optima fluorescent plate reader) during treatment with bovine β trypsin (Sigma Chemicals), PAR-2 activating peptide SLIGKV (AusPep) and iKLK4 with or without SFTI-FCQR.

### Molecular Modelling

All modelling and superpositions were performed using COOT [43]. Residue packing and interatomic clashes were visualized and monitored in COOT using the programs REDUCE and PROBE [44]. First, a KLK4-SFTI complex was modelled by superimposing KLK4 (PDB id 2BDG, chain A) with the trypsin chain of a trypsin-SFTI complex (PDB id 1 SFI) and removing any unfavourable interactions by conjugate gradient energy minimization with CNS [45]. Backbone atoms of both the protease and SFTI-FCQR molecule were fixed, in light of the rigidity of the inhibitor and structural conservation of KLK4 with trypsin, while sidechains were subject to harmonic restraints. Molecular surfaces were created using CCP4MG [46] and colour coded according to electrostatic potential (calculated by the Poisson-Boltzmann solver within CCP4MG). The probe radius used was 1.4 Å. The resulting molecular surfaces were rendered using POVRAY (see the website at www.povray.org). PyMol (see the website at http://pymol.sourceforge.net/newman/user/toc.html) was used to produce.

Molecular dynamics simulations of SFTI variants was performed using the software Nanoscale Molecular Dynamics (NAMD) 2.6 (Phillips et al., J. Comp. Chem. 26:1781-1802, 2005) with the following parameters: Cell extension 12; cutoff 11.0; switching on; switchdist 9.0; pairlistdist 12.5. Simulations consisted of three 1 ns equilibration phases followed by a 25 ns data collection phase. The efficacy of simulated inhibitor binding was evaluated as an average binding energy over the 25 ns simulations calculated in YASARA using macro atomisation. Poorly binding residues in the SFTI variants were identified by total absolute movement relative to KLK4 over time. SFTI variants where substitutions of residues resulted in greatly reduced total absolute movement relative to KLK4 over time was assayed against KLK4 to determine IC₅₀ values.

### Results

### Rational Design of a Small Molecular Weight Inhibitor

The design strategy consisted of three discrete steps. Initially, a focused tetrapeptide library of para-nitroanilide substrates was screened to determine the optimal substrate for KLK4, which revealed that the sequence FVQR outperformed previous candidate inhibitors. This substrate was then used to design an intermediate peptide aldehyde inhibitor to verify the hypothesis that efficient substrates for KLK4 could also be used as the basis of efficient inhibitors. Finally, the optimal tetrapeptide sequence was substituted into the SFTI scaffolding to produce an inhibitor with an amidolytic Kᵢ of 3.25±1.60 nM.

### KLK4 has a Marked Preference for a Phenyl Group at P4 and FVQR is its Optimal Tetrapeptide Substrate

Enhancement of the potency and selectivity of inhibition of the wild-type SFTI towards KLK4 required a detailed knowledge of the enzyme's ligand-binding determinants. Accordingly, the enzyme active site was probed with a sparse matrix library of chromogenic peptide substrates. Results from the library screen are summarized in FIG. 1. The substrate FVQR-pNa was hydrolysed most efficiently (6.7 times average rate) followed by FTQR-pNa and IVQR-pNa (4.4 and 4.3 times average rates respectively). Significantly, IVQR had previously been regarded as the optimal tetrapeptide substrate for KLK4.

KLK4 has previously been shown to have highest amidolytic activity towards the standard tripeptide substrate Bz-FVR-pNA out of all experimentally tested colorimetric peptide substrates [20]. Therefore, this substrate was used as a benchmark when determining KLK4′s enzymatic activity towards FVQR-pNa. A number of peptide pNA substrates were synthesised, purified and further characterized. Given the preference for phenyl and bulky hydrophobic residues at the N-terminus of the peptide substrates as determined in these studies, the contribution of the benzoyl capping group of Bz-FVR-pNA was examined to its apparent preference as an amidolytic substrate for KLK4. Accordingly, an acetylated variant, Ac-FVR-pNA, was evaluated, and since IVQR scored second best in the sparse matrix library and IVQR had previously been suggested to be the optimal KLK4 cleavage sequence [32], IVQR-pNA was also included. In addition, FVMR-pNA was also synthesized to assess the importance of the preference observed for the polar glutamine residue in the P2 position, by replacing it with a more hydrophobic methionine residue. Kinetic data for these substrates are summarized in Table 1. KLK4 showed highest amidolytic activity towards FVQR-pNA of all chromogenic substrates examined. Replacing the N-terminal benzoyl group of Bz-FVR-pNA with an acetyl group resulted in a 47% reduction of k_{cat}, while replacing Phe with a Val at the P4 position resulted in an 87% reduction of k_{cat}, indicating an important role of the benzene ring in the interaction with KLK4 at the P4 position. The considerable reduction in k_{cat} as a result of replacing Gln with Met in the S2 or P2 position showed that the polarity of the Gln sidechain plays an important role in the rate of hydrolysis of this substrate. As a result of this, the sequence FVQR was used as the basis of inhibitor design rather than IVQR and FQQR as predicted by Matsumura et al. [20] and Debela et al. [32], or the commercially available tripeptide benzoyl FVR-pNA that until now has been considered the substrate with highest k_{cat}.

### Peptide Aldehydes Based on Substrate Sequences are Inhibitors of KLK4

It was hypothesised that sequences that were efficient substrates for KLK4 would be complementary to the S1-S4 subsites of the enzyme's catalytic centre (numbering of the enzyme's catalytic centre is according to [40]). Consequently, a series of peptide aldehydes based on the optimal sequence from the sparse matrix peptide library, the best substrate predicted by PS-SCL (IVQR) and an aldehyde based on Bz-FVR-pNA were created. The aldehyde groups on these compounds mimic the transition state of the amidolytic reaction, producing molecules that inhibit proteolysis. The two library-based aldehydes inhibited KLK4 amidolytic activity at μM levels, while Bz-FVR aldehyde did not inhibit KLK4 in this range. FVQR aldehyde was the most efficient inhibitor by an order of magnitude with an IC₅₀ of 10.8±1.3 μM compared to 103.3±2.8 μM for IVQR.

### KLK4 is Inhibited By Wild-Type SFTI

Given that trypsin and KLK4 share significant structural (rmsd=1.1 Å over 223 Cα atoms) and sequence similarity (40% overall and 73% identity within the area adjacent to SFTI in the crystal structure 1SFI.pdb) and are both trypsin-like serine proteases, SFTI should be able to inhibit KLK4 in addition to trypsin. Assays with the amidolytic standard KLK4 substrate Bz-FVR-pNA showed that the wild-type inhibitor could indeed block KLK4 activity with an IC₅₀ of 221.4±1.10 nM.

### SFTI-FCQR is a Potent and Specific Inhibitor of KLK4

KLK4 showed a marked preference for the sequence FVQR as a substrate for cleavage and as an aldehyde inhibitor. Accordingly, this sequence was substituted into the SFTI backbone to increase the scaffold's potency of inhibition. The variant SFTI molecule was designed such that the key P1 residue (Lys5) was replaced by arginine; with glutamine replacing Thr4. Cys3 was not substituted given the pivotal role of this residue in the structural stability of SFTI, while Arg2 was replaced by phenylalanine Inhibitory potency and specificity of the resulting variant (SFTI-FCQR) was evaluated against KLK4. Enzyme velocities were monitored with varying inhibitor concentration in the range of 2.0 μM-1 nM to determine IC₅₀ (Bz-FVR-pNA; 0.12 mM) and k, (FVQR-pNA) values. SFTI-FCQR was determined to be a potent KLK4 inhibitor with an IC₅₀ of 7.97±1.08 nM and a kᵢ of 3.59±1.08 nM (Table 2).

Wild-type SFTI displays promiscuous inhibition, blocking the activity of diverse proteases including suppressor of tumourgenesis 14 ST14/matriptase and cathepsin G in addition to trypsin. Accordingly, a panel of serine proteases, including trypsin, thrombin, α-chymotrypsin, Streptomyces griseus type XIV protease, plasminogen protease, and suppressor of tumourgenesis 14 ST14/matriptase, were assayed to assess their inhibition by wild-type SFTI and SFTI-FCQR. SFTI-FCQR did not inhibit any of these proteases, with the exception of trypsin, which was only inhibited in the micromolar range (IC₅₀ 4.064±1.088 μM; Table 2).

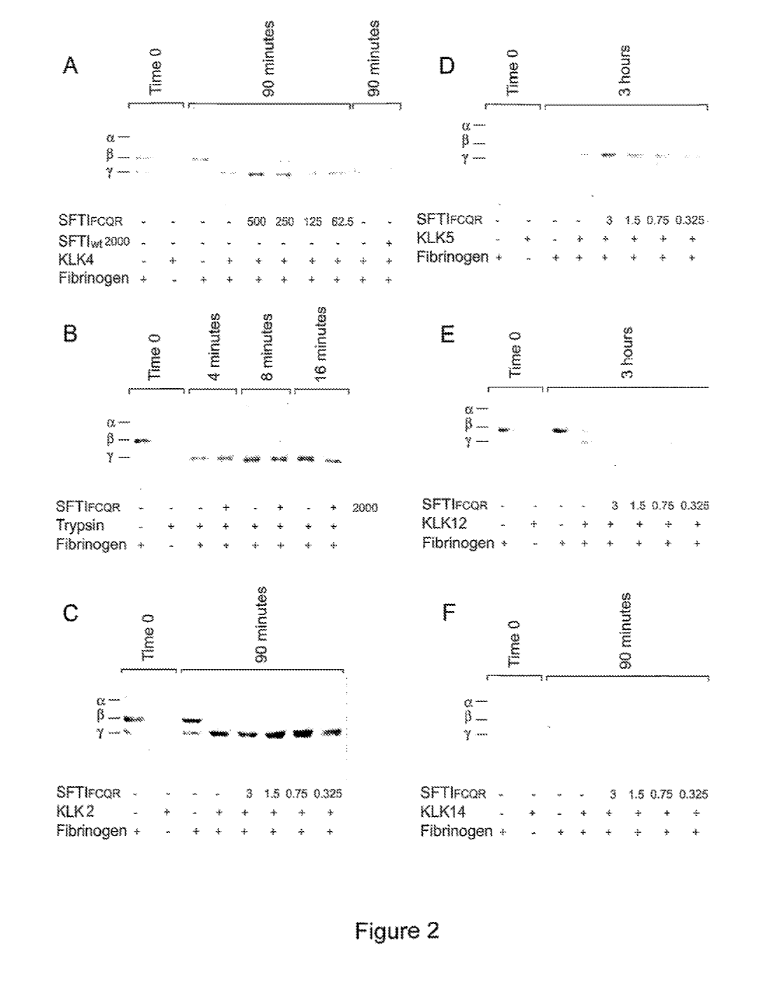

While inhibition of serine proteases is most usually measured using small peptide and ester substrates, the important biological activities of the enzymes are the result of protein proteolysis. Given this, both the wild type and FCQR variant inhibitors were assayed for their ability to inhibit KLK4 proteolysis of fibrinogen, a known substrate for the enzyme [17]. Surprisingly, inhibition of fibrinogen proteolysis did not reflect inhibition of peptide-pNA hydrolysis. The FCQR variant was found to be a potent inhibitor of fibrinogen proteolysis by KLK4, with complete inhibition at 250 nM (FIG. 2A). Unexpectedly, no inhibition of proteolysis by KLK4 could be detected for the wild-type inhibitor even at a concentration of 2 μM, compared to a substrate concentration of 0.8 μM and an enzyme concentration of 2.5 nM (FIG. 2A), a marked contrast to the inhibition of amidolytic activity. Similarly, the SFTI-FCQR variant showed no inhibition of tryptic digestion of fibrinogen up to a concentration of 2 μM (FIG. 2B), or with KLK2, 5, 12, and 14 (see FIGS. 2C-2F).

SFTI-FCQR Maintains its Inhibitory Activity with a Half Life of Four Days in Tissue Culture

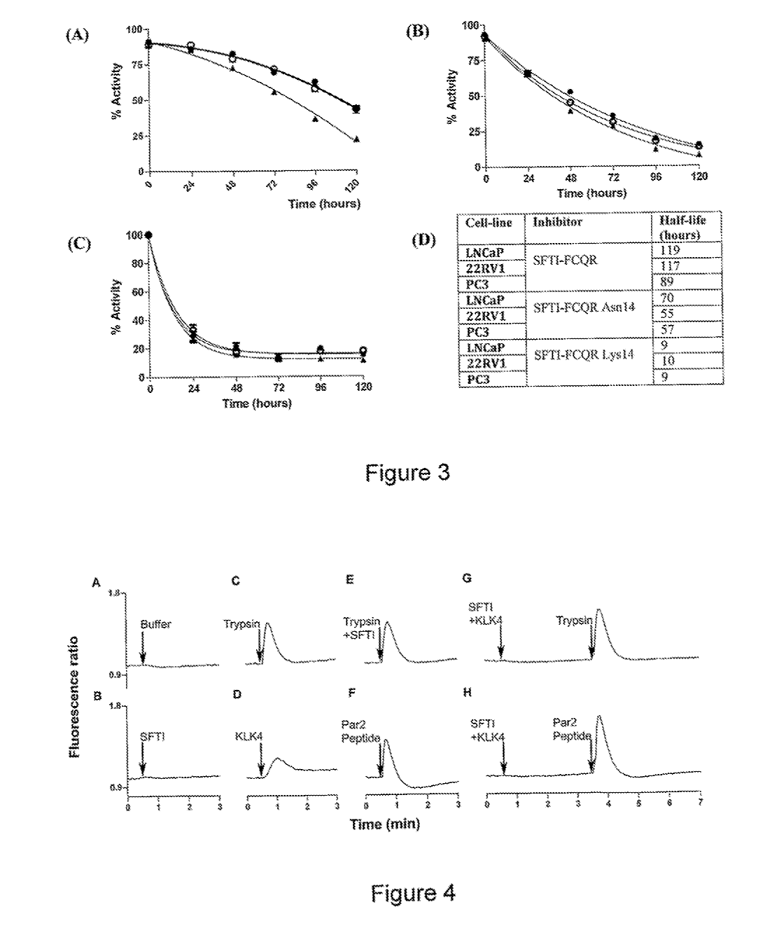

To gauge the half life of SFTI-FCQR in a cellular environment, STFI-FCQR was incubated at a concentration of 1 μM with LNCaP, 22RV1 and PC3 prostate cancer cell lines. Residual SFTI-FCQR inhibitory activity was measured in tissue culture supernatants that had been boiled and centrifuged to remove serum derived protease inhibitors. Assaying a 1:5 dilution of tissue culture supernatant (200 nM concentration at time=0) against recombinant KLK4 gave complete inhibition even after a week of contact with prostate cancer cells. Dilution by a further 20 fold was required before any appreciable reduction of inhibitory activity could be observed. Half lives for inhibitor incubated with LNCaP and 22RV1 were similar (FIGS. 3; t_{1/2}=119 hr and 117 hr respectively), while the decline in inhibition was accelerated in PC3s (t_{1/2}=89 hr). Decay of inhibition followed a secondary polynomial relationship with the exponential component being concurrent for all three cell lines while the linear component was nearly five times greater in the presence of PC3s. This difference may correlate with the higher metabolic rate observed in this cell line compared with LNCaP and 22RV1 as determined by WST-1 assays. These halflife measurements show that SFTI-FCQR enjoys considerable stability in a cellular milieu.

### SFTI-FCQR Blocks KLK4 Stimulated Calcium Flux in Cell Based Assays

Recent work by Ramsay et al. [41] and Mize et al [47] has demonstrated that KLK4 is able to stimulate the protease activated receptors PAR-1 and PAR-2, causing release of intracellular calcium, phosphorylation of ERK1/2 and ultimately increasing rates of proliferation in DU145 prostate cancer cells. These effects could be blocked by the non-specific protease inhibitor aprotinin. Consequently, SFTI-FCQR's effect on KLK4 stimulated calcium release in cell based assays was assessed. The inhibitor showed extremely robust blockade of calcium release when stimulated by KLK4 treatment in the presence of 1 μM SFTI-FCQR (FIG. 4). Furthermore, the inhibitor was specific, blocking KLK4 only and being permissive for calcium release by both trypsin and PAR activating peptides.

### Modelling a KLK4-SFTI-FCQR Complex

To gain a molecular understanding of the behaviour of the substituted SFTI variant, a predictive modelling of an SFTI-FCQR/KLK4 complex was carried out using the SFTI/trypsin structure 1 SFI and the KLK4 structure 2BDG as a starting point. Trypsin and KLK4 superimpose closely, with an RMSD of 1.1 Å over 223 Cα atoms. Active site regions superimpose extremely well, with the exception of residues that form the S4 subsite: residues 95-98 (the “99 loop”) fold back, away from the active site of KLK4 in comparison to trypsin, resulting in differences of up to 3 A; residues in the 164-180 loop also display large differences. These differences have two important consequences. First, the sidechains of L98, L99, and L175 in KLK4 move towards the S4 pocket, making it more hydrophobic. Second, the negative S2 pocket, with D102 (catalytic) at its base, widens and deepens, making it accessible by the substituted Gln in the FCQR SFTI variant, which can H-bond with residues lining the pocket.

Structural similarities between KLK4 and trypsin were reflected in the predicted structure for the FCQR-SFTI variant. The variant SFTI was modelled by simple substitution of the wild type structure coupled with rotamer-based side chain modelling and energy minimisation. This produced only subtle atomic shifts that relieved close atomic contacts. Each substitution and subsequent structural changes in the context of KLK4-trypsin structural differences is discussed herein. Particular attention is drawn to the net balance of hydrogen bonds and non-polar interactions at the site of substitution.

K5R (Pᵢ): The most statistically-preferred Arg rotamer correlates well with the wild type Lys sidechain conformation. Energy minimization relieves a close contact of K5 with S190 in the trypsin-SFTI complex and the modelled KLK4 R5 side chain forms 2 hydrogen bonds with D189 at the base of the electronegative 51 pocket. However, an intramolecular hydrogen bond with S10 is lost.

T4Q (P₂): Substitution of the Thr sidechain in SFTI removes 2 intramolecular hydrogen bonds, with S6 and I10 of SFTI. However, T4 does not hydrogen-bond with the protease. Modelling a Gln at this position (using 2nd most statistically preferred rotamer) in the FCQR-SFTI allows 2 hydrogen bonds between its sidechain carbonyl group and the protease (catalytic H57 sidechain and 5214 mainchain). In addition, when adopting this conformation, the relatively larger side chain of Gln fits snugly into the S2 pocket that is wider in KLK4 compared to trypsin.

R2F (P₄): The sidechain of R2 in SFTI forms a hydrogen bond with the backbone of N97 of trypsin. A Phe at this position in FCQR-SFTI can be modelled satisfactorily using the most preferred rotamer, and makes non-polar interactions with the sidechains of L175 and F215 of the protease. In response to substitution, small movements of the sidechain of D14 of the inhibitor (in order to remove unfavourable close contacts) result in the loss of a hydrogen bond between D14 and the SFTI backbone.

The preferences for residues at P1, P2 and P4 can be explained largely by the physiochemical nature of each subsite in the modelled complex, with the exception of the poor preference for Tyr at P4, compared to Phe. Indeed, in the current model a tyrosine could be positioned in a similar orientation to the Phe, with neighbouring polar groups positioned to form hydrogen bonds with its OH moiety. The poor preference for Tyr, however, can be rationalised by assuming an alternative sidechain rotamer that positions its sidechain deeper into the hydrophobic core created by residues L99, L175 and F215. The absence of polar hydrogen-bonding partners for the OH moiety would destabilise this residue, rationalising the experimental data presented here. However, modelling Tyr at this position produces several close contacts with neighbouring residues that can only be relieved by relatively large structural rearrangements in the surroundings.

### SFTI-FCQR Arrests Primary Tumours in In Vivo Assays

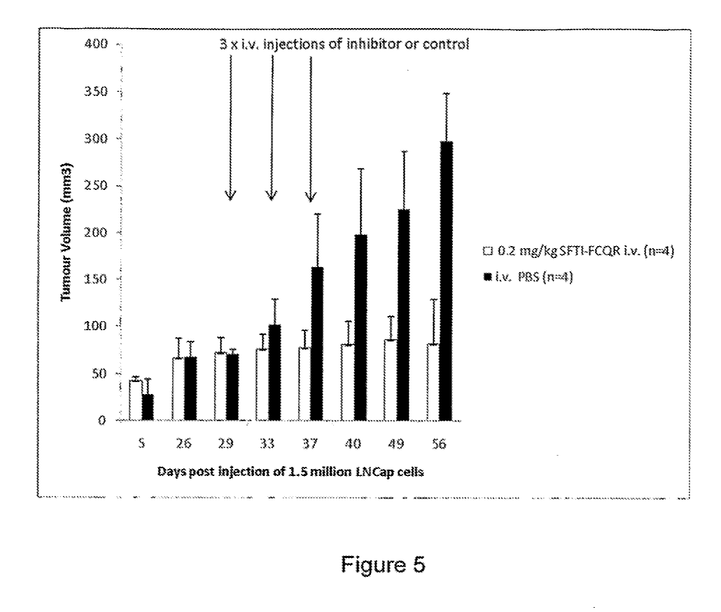

Nude mice were injected with 1.5×10⁶ LNCaP prostate cancer cells stably expressing an exogenous luciferase reporter gene and imaged over a period of 56 days. Tumour growth was measured by injecting mice with luminol (a luminescent substrate for luciferase) and the resulting light emissions were quantified using a xenogen in vivo imaging system. When tumour specific growth rate was less than 2%/day, administration of 3×i.v. doses of 0.2 mg/kg SFTI-FCQR arrested tumour growth rate in comparison to controls (FIG. 5).

### SFTI-FCQR Variants as Inhibitors of KLK4

SFTI-FCQR variants were produced by using molecular modelling to pinpoint opportunities for improved affinity for the target protease, while at the same time maintaining the molecule's selectivity. Table 3 illustrates some of the preferred amino acid substitutions at various positions within SFTI. One such variant identified was SFTI-FCQR Ala9, an additional potent and specific inhibitor of KLK4.

It was found that KLK4 has a unique hydrogen bond acceptor in close proximity to aspartate 14 of SFTI. To increase hydrogen bonding between the protease and inhibitor a series of variants designed to engage this hydrogen bond acceptor were assayed, including SFTI-FCQR Asn14 and SFTI-FCQR Lys14. Substitution of asparagine at this position to produce SFTI-FCQR Asn14 yielded a potent and specific inhibitor with respect to the closest kallikrein in terms of sequence homology (i.e., KLK14).

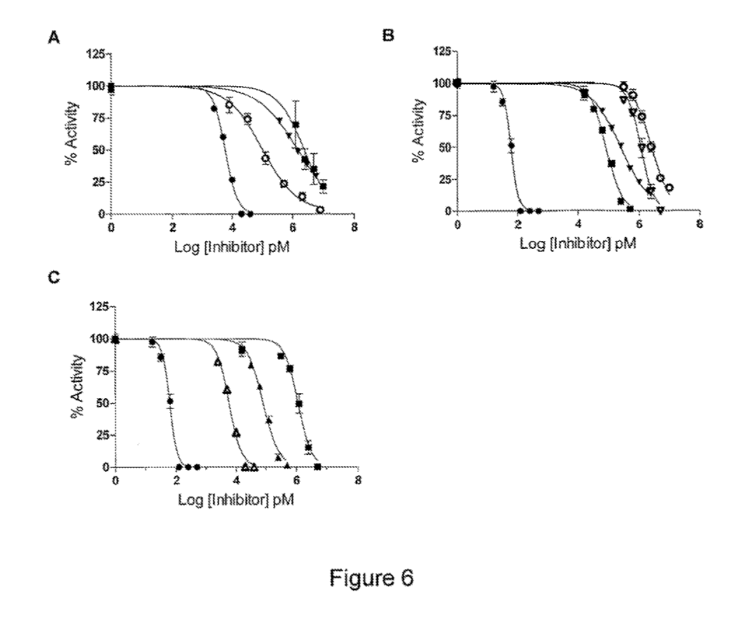

Both chromogenic substrates and protein substrates were used to assess the potency and specificity of SFTI-FCQR Asn14. Chromogenic substrate (FVQR-paranitroanilide) indicated an IC₅₀ of 0.063±0.002 nM for the inhibitor against KLK4 (Table 2). This compared with an IC_{50 0}f 250.8±6.65 nM when assayed against the closely related KLK14 (using its favoured substrate: GSLR-paranitroanilide), indicating that the asparagine substitution had not affected SFTI-FCQR Asn14′s selectivity. SFTI-FCQR Lys 14 had considerably lower potency against KLK4, with an IC₅₀ nearly three orders of magnitude higher (Table 2). IC₅₀ Values for SFTI and several of the SFTI variants with various proteases was undertaken; the results are shown in FIG. 6.

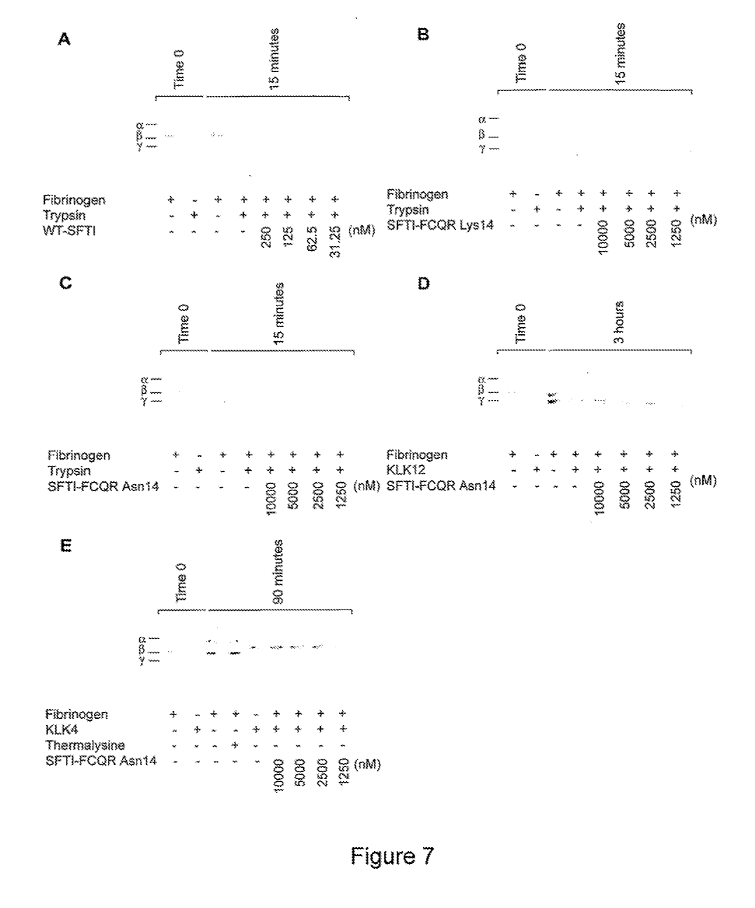

As discussed herein, during the course of studies on the original SFTI-FCQR variant, it was found that inhibition of protein proteolysis, which is the in vivo target of proteolysis inhibition, did not follow protein proteolysis. Accordingly, a series of analyses using the protein fibrinogen as a substrate for proteolytic activity was undertaken. Data from theses digests again demonstrated the specificity and potency of SFTI-FCQR Asn14 (FIG. 7).

To assess the stability of SFTI-FCQR Asn14 and to gain preliminary PK data, the inhibitor was incubated with a number of different prostate cancer cell lines growing in serum containing medium. As with the original SFTI-FCQR inhibitor, inhibition by SFTI-FCQR Asn14 had a half life in the order of days, depending on cell type used for incubation (FIG. 3). Interestingly, the lysine 14 variant had a curtailed half life compared with both SFTI-FCQR Asn14 and SFTI-FCQR (FIG. 3). These half lives compare very favourably with those of other protease inhibitors including ritonavir (3.8 hours).

### Discussion

A peptide sequence that performs well as a substrate for a target protease can be substituted into the naturally occurring SFTI scaffold to produce an inhibitor with a nanomolar Kᵢ and reduced ability to inhibit the proteases thrombin and suppressor of tumourgenesis 14 ST14/matriptase (by three orders of magnitude). Additionally, inhibition of peptide cleavage by a protease does not necessarily reflect inhibition of cleavage of protein substrates. Furthermore, the SFTI scaffold exhibits extreme robustness towards degradation in a cellular context (even in PC3 cells with very high metabolic activity), refuting previous studies which focused on engineering the supposedly labile disulphide bond.

It has been previously shown that PAR1 and 2 are upregulated in prostate cancer cells [14] and that PAR1 and 2 activity plays a role in proliferation and migration of prostate cancer cells [17]. Accordingly, PAR activity is an attractive target for chemotherapeutic intervention. As disclosed herein, the inventors have inhibited the stimulation of PAR2 by its putative cognate in vivo protease, KLK4, which is also upregulated during prostate cancer progression. PAR2 inhibition was assessed in cell based assays, which demonstrated the potency of inhibition by a SFTI variant by complete inhibition of calcium flux at a concentration of 1 mM. Significantly, the inhibitor had no effect on trypsin stimulated calcium flux or on PAR2 activity elicited by an exogenously added PAR2 activating peptide. The inhibitory activity of SFTI-FCQR and other SFTI variants as disclosed herein (e.g., SFTI-FCQR Asn14) can form the basis of a novel treatment for prostate cancer.

The SFTI scaffold represents an excellent platform for drug design. With only 14 amino acids and a molecular mass of 1,513 Da, SFTI is the smallest, as well as the most potent (β-trypsin: Kᵢ=0.1 nM) naturally occurring serine protease inhibitor [16]. Comparison of the structure of SFTI in complex with β-trypsin and in solution [32] indicates that the peptide's conformation does not change markedly upon binding to the protease, reflecting stabilization of the inhibitor both by cyclisation and a bisecting disulphide bond. Important determinants of disulfide bond stability include neighbouring residues of aromatic and amino side chains near the bond greatly reducing and increasing the reactivity respectively [42, 48]. The disulfide bond in SFTI-FCQR is surrounded by Phe2 and G1n4 on one side and Ile10 and Phe12 on the other side. Since Gln4 is extended as far as sterically possible away from the disulfide bond due to a hydrogen bond with Ser6, the disulfide bond is likely to be quite stable as revealed by its extended half life in tissue culture based assays.

Inhibitors resulting from these studies have both enhanced inhibitory potency and specificity. The importance of inhibitor specificity has been highlighted by recent failures in clinical trials of a series of metalloproteinase (MMP) inhibitors which were developed in an attempt to block MMP mediated digestion of extra cellular matrix ECM components and halt tumour progression [49]. Some of these entered phase III clinical trials [50], although most have been terminated either due to no or negative survival benefits [51].

The role of specificity as well as affinity is further attested to by the failure of the wild-type SFTI to inhibit digestion of fibrinogen by KLK4. Hence, although the wild-type SFTI may bind with high affinity to KLK4, it has lower specificity than the FCQR variants described herein. This is especially important in view of the pivotal role that fibrinogen remodelling plays in maintenance of the extracellular matrix and progression of cancer. Furthermore, although wild-type SFTI may compete efficiently with a short peptide for the enzyme's active site, it is completely ineffective when competing with a larger protein binding with higher affinity and specificity when compared to FCQR variants, with their superior selectivity as attested to by its lack of inhibition of trypsin proteolysis.

In conclusion, inhibitory activity of a naturally occurring cyclic molecule has been redirected through substrate-guided and computer-assisted design. The molecules of the invention exhibit greatly increased potency towards the intended target, an enzyme with an important potential role in the development and progression of prostate cancer.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

All computer programs, algorithms, patent and scientific literature referred to in this specification is incorporated herein by reference in their entirety.

TABLE 1
K_{M}, k_{cat} and Catalytic Efficiency
(k_{cat}/K_{M}) for para-Nitroanilide substrate

| Substrate | K_{M} (μM) | k_{cat} (sec⁻¹) | k_{ca}t/K_{M} (M⁻¹s⁻¹) |
|---|---|---|---|
| Bz-FVR-pNA | 42.5 ± 5.9 | 0.57 ± 0.031 | 1.34 ± 0.15 × 10⁴ |
| Ac-FVR-pNA | 194.9 ± 41.4 | 0.30 ± 0.032 | 1.54 ± 0.24 × 10³ |
| FVMR-pNA | 74.7 ± 10.3 | 0.38 ± 0.019 | 5.09 ± 0.15 × 10⁴ |
| IVQR-pNA | 183.1 ± 12.14 | 0.63 ± 0.021 | 3.44 ± 0.07 × 10³ |
| FVQR-pNA | 679.9 ± 113.1 | 4.71 ± 0.47 | 6.93 ± 0.19 × 10⁴ |

TABLE 2
Inhibitory Properties of Wild Type SFTI and SFTI-FCQR

| | | | | | Substrate |
|---|---|---|---|---|---|
| Enzyme | Inhibitor | Kᵢ (nM) | Substrate | IC₅₀ (nM) | (0.2 mM) |
| KLK4 | SFTI-1 | — | — | 221.4 ± 1.10 | Bz-FVR-pNA |
| | SFTI-FCQR | 3.59 ± 0.28 | FVQR-pNA | 7.97 ± 1.08 | FVQR-pNA |
| | SFTI-FCQR Lys14 | — | — | 98.94 ± 4.09 | FVQR-pNA |
| | SFTI-FCQR Asn14 | — | — | 0.063 ± 0.002 | FVQR-pNA |
| | SFTI-FCQR Ala9 | — | — | 1142 ± 76 | FVQR-pNA |
| KLK5 | SFTI-FCQR | — | — | 2348 ± 721 | Bz-PFR-pNA |
| iKLK14 | SFTI-FCQR | — | — | 1506 ± 37.1 | GSLRpNA |
| | SFTI-FCQR Asn14 | | | 250.8 ± 6.65 | GSLRpNA |
| β-Trypsin | SFTI-1 | 0.1^{a} | BAPNA | — | — |
| | SFTI-FCQR | — | — | 4064 ± 1088 | BAPNA |
| | SFTI-FCQR Lys14 | — | — | Inhibitor degraded | BAPNA |
| | SFTI-FCQR Asn14 | — | — | 2579 ± 507.7 | BAPNA |
| Thrombin | SFTI-1 | 136^{a} | Unknown | — | — |
| | | 5050^{b} | N-t-Boc-LRR-AMC | — | — |
| | SFTI-FCQR | — | — | >10,000 | Bz-FVR-pNA |
| | SFTI-FCQR Lys14 | — | — | >10,000 | Bz-FVR-pNA |
| | SFTI-FCQR Asn14 | — | — | >10,000 | Bz-FVR-pNA |
| Matriptase | SFTI-1 | 0.92^{b} | N-t-Boc-QAR-AMC | — | — |
| | SFTI-FCQR | — | — | >10,000 | Bz-FVR-pNA |
| | SFTI-FCQR Lys14 | — | — | >10,000 | Bz-FVR-pNA |
| | SFTI-FCQR Asn14 | — | — | >10,000 | Bz-FVR-pNA |
| α-Chymotrypsin | SFTI-1 | 2,300 ± 100^{c} | N-succinyl-AAPP-pNA | 1,800 ± 110 | W-pNA |
| | SFTI-FCQR Lys14 | — | — | >10,000 | W-pNA |
| | SFTI-FCQR Asn14 | — | — | >10,000 | W-pNA |
| | SFTI-FCQR | — | — | >10,000 | W-pNA |
| Plasminogen | SFTI-FCQR | — | — | >10,000 | L-pNA, BAPNA |
| Protease | SFTI-FCQR | — | — | >10,000 | L-pNA, BAPNA |
| | SFTI-FCQR Lys14 | — | — | >10,000 | L-pNA |
| | SFTI-FCQR Asn14 | — | — | >10,000 | L-pNA |

^{a}Luckett, S., Garcia, R. S., Barker, J. J., Konarev, A. V., Shewry, P. R., Clarke, A. R. & Brady, R. L. (1999) J. Mol. Biol., 290, 525-33.
^{b}Ya-Qiu Long, Sheau-Ling Lee, Chen-Yong Lin, Istvan J. Enyedy, b Shaomeng Wang, b Peng Li, a Robert B. Dicksonb and Peter P. Rollera, Bioorganic & Medicinal Chemistry Letters 11 (2001) 2515-2519.
^{c}Descours, A., Moehle, K., Renard, A. & Robinson, J. A. (2002) Chembiochem, 3, 318-23.

TABLE 3
Amino Acid Substitutions at Various Positions Within SFTI

| | | Preferred | Non-preferred |
|---|---|---|---|
| Position | Current AA | Substitutions | Substitutions |
| 14 | Aspartic acid | Asparagine | Serine |
| | | Lysine | Threonine |
| | | Arginine | Cysteine |
| | | Tryptophan | Methionine |
| | | | Other hydrophobic |
| | | | amino acids |
| 7 | Isoleucine | Lysine | |
| | | Valine | |
| | | Phenylalanine | |
| | | Other hydrophobic | |
| | | amino acids | |
| 10 | Isoleucine | Phenylalanine | |
| 1 | Glycine | Serine | |
| | | Threonine | |
| 9 | Proline | Alanine | |

### REFERENCES

1. Abel, P. D., and Lalani, E.-N. (2003) Prostate cancer: clinical and scientific aspects: bridging the gap Prostate cancer: clinical and scientific aspects: bridging the gap, Imperial College Press, London
2. Borgono, C. A., and Diamandis, E. P. (2004) The emerging roles of human tissue kallikreins in cancer. Nature Reviews Cancer, 4; 876-890
3. Yousef, G. M., Magklara, A., Chang, A., Jung, K., Katsaros, D., and Diamandis, E. P. (2001) Cloning of a New Member of the Human Kallikrein Gene Family, KLK14, Which Is Down-Regulated in Different Malignancies. Cancer Research, 61; 3425-3431
4. Yousef, G. M., Polymeris, M.-E., Yacoub, G. M., Scorilas, A., Soosaipillai, A., Popalis, C., Fracchioli, S., Katsaros, D., and Diamandis, E. P. (2003) Parallel Overexpression of Seven Kallikrein Genes in Ovarian Cancer. Cancer Research, 63; 2223-2227
5. Dong, Y., Kaushal, A., Bui, L., Chu, S., Fuller, P. J., Nicklin, J., Samaratunga, H., and Clements, J. A. (2001) Human kallikrein 4 (KLK4) is highly expressed in serous ovarian carcinomas. Clinical Cancer Research, 7; 2363-2371
6. Yousef, G. M., Magklara, A., and Diamandis, E. P. (2000) KLK12 is a novel serine protease and a new member of the human kallikrein gene family-differential expression in breast cancer. Genomics, 69; 331-341
7. Magklara, A., Scorilas, A., Stephan, C., Kristiansen, G. O., S., H., Jung, K., and Diamandis, E. P. (2000) Decreased concentrations of prostate-specific antigen and human glandular kallikrein 2 in malignant versus nonmalignant prostatic tissue. Urology, 56; 527-532
8. Simmer, J. P., Ryu, O. H., Qian, Q., Zhang, C., Cao, X., and Sun, X. (2000.) Chemistry and biology of mineralized tissues: proceedings of the sixth international conference Chemistry and biology of mineralized tissues: proceedings of the sixth international conference(Goldberg, M. a. R., C., Ed.), American Academy of Orthopaedic Surgeons, Vittel, France
9. Stephenson, S. A., Verity, K., Ashworth, L. K., and Clements, J. A. (1999) Localization of a new prostate-specific antigen-related serine protease gene, KLK4, is evidence for an expanded human kallikrein gene family cluster on chromosome 19q13.3-13.4. Journal of Biological Chemistry, 274; 23210-23214
10. Nelson, P. S., Gan, L., Ferguson, C., Moss, P., Gelinas, R., Hood, L., and Wang, K. (1999) Molecular cloning and characterization of prostase, an androgen-regulated serine protease with prostate-restricted expression. Proceedings of the National Academy of Science, 96; 3114-3119
11. Yousef, G. M., Obiezu, C. V., Luo, L.-Y., Black, M. H., and Diamandis, E. P. (1999) Prostase/KLK-L1 Is a New Member of the Human Kallikrein Gene Family, Is Expressed in Prostate and Breast Tissues, and Is Hormonally Regulated. Cancer Research, 59; 4252-4256
12. Dong, Y., Bui, L. T., Odorico, D. M., Tan, O. L., Myers, S. A., Samaratunga, H., Gardiner, R. A., and Clements, J. A. (2005) Compartmentalized expression of kallikrein 4 (KLK4/hK4) isoforms in prostate cancer: nuclear, cytoplasmic and secreted forms. Endocrine Related Cancer, 12; 875-889
13. Davidson, B., Xi, Z., and Saatcioglu, F. (2007) Kallikrein 4 is expressed in malignant mesothelioma—further evidence for the histogenetic link between mesothelial and epithelial cells. Diagnostic Cytopathology, 35; 80-84
14. Veveris-Lowe, T. L., Lawrence, M. G., Collard, R. L., Bui, L., Herington, A. C., Nicol, D. L., and Clements, J. A. (2005) Kallikrein 4 (hK4) and prostate-specific antigen (PSA) are associated with the loss of E-cadherin and an epithelial-mesenchymal transition (EMT)-like effect in prostate cancer cells. Endocrine Related Cancer, 12; 631-643
15. Xi, Z., Klokk, T. I., Korkmaz, K., Kurys, P., Elbi, C., Risberg, B., Danielsen, H., Loda, M., and Saatcioglu, F. (2004) Kallikrein 4 is a predominantly nuclear protein and is overexpressed in prostate cancer. Cancer Research, 64; 2365-2370
16. Gao, J., Collard, R. L., Bui, L., Herington, A. C., Nicol, D. L., and Clements, J. A. (2007) Kallikrein 4 is a potential mediator of cellular interactions between cancer cells and osteoblasts in metastatic prostate cancer. Prostate, 67; 348-360
17. Obiezu, C. V., Michael, I. P., Levesque, M. A., and Diamandis, E. P. (2006) Human kallikrein 4: enzymatic activity, inhibition, and degradation of extracellular matrix proteins. Biological Chemistry, 387; 749-759
18. Takayama, T. K., Fujikawa, K., and Davie, E. W. (1997) Characterization of the Precursor of Prostate-specific Antigen. Journal of Biological Chemistry, 272; 21582-21588
19. Takayama, T. K., McMullen, B. A., Nelson, P. S., Matsumura, M., and Fujikawa, K. (2001) Characterization of hK4 (prostase), a prostate-specific serine protease: activation of the precursor of prostate specific antigen (pro-PSA) and single-chain urokinase-type plasminogen activator and degradation of prostatic acid phosphatase. Biochemistry, 40; 15341-15348
20. Matsumura, M., Bhatt, A. S., Andress, D., Clegg, N., Takayama, T. K., Craik, C. S., and Nelson, P. S. (2005) Substrates of the prostate-specific serine protease prostase/KLK4 defined by positional-scanning peptide libraries. The Prostate, 62; 1-13
21. Nagano, T., Oida, S., Ando, H., Gomi, K., Arai, T., and Fukae, M. (2003) Relative levels of mRNA encoding enamel proteins in enamel organ epithelia and odontoblasts. Journal of Dental Research, 82; 982-986
22. Hu, J. C.-C., Zhang, C., Sun, X., Yang, Y., Cao, X., Ryu, O., and Simmer, J. P. (2000) Characterization of the mouse and human PRSS17 genes, their relationship to other serine proteases, and the expression of PRSS17 in developing mouse incisors. Gene, 251; 1-8
23. Pope, S. N., and Lee, I. R. (2005) Yeast two-hybrid identification of prostatic proteins interacting with human sex hormone-binding globulin. Journal of Steroid Biochemistry and Molecular Biology, 94; 203-208
24. Beaufort, N., Debela, M., Creutzburg, S., Kellermann, J., Bode, W., Schmitt, M., Pidard, D., and Magdolen, V. (2006) Interplay of human tissue kallikrein 4 (hK4) with the plasminogen activation system: hK4 regulates the structure and functions of the urokinase-type plasminogen activator receptor (uPAR). Biological Chemistry, 387; 217-222
25. Hart, P. S., Hart, T. C., Michalec, M. D., Ryu, O. H., Simmons, D., Hong, S., and Wright, J. T. (2004) Mutation in kallikrein 4 causes autosomal recessive hypomaturation amelogenesis imperfecta. Journal of Medical Genetics, 41; 545-549
26. Hu, J. C., Sun, X., Zhang, C., Liu, S., Bartlett, J. D., and Simmer, J. P. (2002) Enamelysin and kallikrein-4 mRNA expression in developing mouse molars. European Journal of Oral Science, 110; 307-315
27. Simmer, J. P., and Hu, J. C. (2002) Expression, structure, and function of enamel proteinases. Connective Tissue Research, 43; 441-449
28. Luckett, S., Garcia, R. S., Barker, J. J., Konarev, A. V., Shewry, P. R., Clarke, A. R., and Brady, R. L. (1999) High-resolution structure of a potent, cyclic proteinase inhibitor from sunflower seeds. Journal of Molecular Biology, 290; 525-533
29. Korsinczky, M. L., Schirra, H. J., Rosengren, K. J., West, J., Condie, B. A., Otvos, L., Anderson, M. A., and Craik, D. J. (2001) Solution structures by 1H NMR of the novel cyclic trypsin inhibitor SFTI-1 from sunflower seeds and an acyclic permutant. Journal of Molecular Biology, 311; 579-591
30. Hilpert, K., Hansen, G., Wessner, H., Volkmer-Engert, R., and Hohne, W. (2005) Complete substitutional analysis of a sunflower trypsin inhibitor with different serine proteases. Journal of Biochemistry (Tokyo), 138; 383-390
31. Marx, U. C., Korsinczky, M. L. J., Schirra, H. J., Jones, A., Condie, B., Otvos, L., Jr., and Craik, D. J. (2003) Enzymatic Cyclization of a Potent Bowman-Birk Protease Inhibitor, Sunflower Trypsin Inhibitor-1, and Solution Structure of an Acyclic Precursor Peptide. Journal of Biological Chemistry, 278; 21782-21789
32. Debela, M., Magdolen, V., Schechter, N., Valachova, M., Lottspeich, F., Craik, C. S., Choe, Y., Bode, W., and Goettig, P. (2006) Specificity Profiling of Seven Human Tissue Kallikreins Reveals Individual Subsite Preferences. Journal of Biolgical Chemistry, 281; 25678-25688
33. Studier, F. W., and Moffatt, B. A. (1986) Use of bacteriophage T7 RNA polymerase to direct selective high-level expression of cloned genes. Journal of Molecular Biology, 189; 113-130
34. Nagai, K., and Thogersen, H. C. (1987) Synthesis and sequence-specific proteolysis of hybrid proteins produced in Escherichia coli. Methods in Enzymology, 153; 461-481
35. Debela, M., Magdolen, V., Grimminger, V., Sommerhoff, C., Messerschmidt, A., Huber, R., Friedrich, R., Bode, W., and Goettig, P. (2006) Crystal structures of human tissue kallikrein 4: activity modulation by a specific zinc binding site. Journal of Molecular Biolology, 362; 1094-1107
36. Chan, W. C. a. W., P. D (ed) (2000) Fmoc Solid Phase Peptide Synthesis: A Practical Approach, 1st Ed. Practical Approach Series.Fmoc Solid Phase Peptide Synthesis: A Practical Approach. Edited by Hammes, B. D., Oxford University Press, New York
37. Abbenante, G., Leung, D., Bond, T., and Fairlie, D. P. (2000) An efficient Fmoc strategy for the rapid synthesis of peptide para-nitroanilides. International Journal of Peptide Research and Therapeutics, 7; 347-351
38. Beynon, R. J., and Bond, J. S. (1989.) Proteolytic enzymes: a practical approach Proteolytic enzymes: a practical approach, IRL Press at Oxford University Press, Oxford, New York
39. Elmar Krieger, T. D., Sander B. Nabuurs, Alexei Finkelstein, Gert Vriend,. (2004) Making optimal use of empirical energy functions: Force-field parameterization in crystal space. Proteins: Structure, Function, and Bioinformatics, 57; 678-683
40. Schechter, I., and Berger, A. (1967) On the size of the active site in proteases. I. Papain. Biochemical and Biophysical Research Communications, 27; 157-162
41. Ramsay, A. J., Reid, J. C., Adams, M. N., Samaratunga, H., Dong, Y., Clements, J. A., and Hooper, J. D. (2008). Prostatic trypsin-like kallikrein-related peptidases (KLKs) and other prostate-expressed tryptic proteinases as regulators of signalling via proteinase-activated receptors (PARs). Biol Chem 389, 653-668
42. Ramsay, A. J., Dong, Y., Hunt, M. L., Linn, M., Samaratunga, H., Clements, J. A., and Hooper, J. D. (2008). Kallikrein-related Peptidase 4 (KLK4) Initiates Intracellular Signaling via Protease-activated Receptors (PARs): KLK4 AND PAR-2 ARE CO-EXPRESSED DURING PROSTATE CANCER PROGRESSION. J. Biol. Chem. 283, 12293-12304
43. Emsley, P., and Cowtan, K. (2004). Coot: model-building tools for molecular graphics. Acta Crystallogr D Biol Crystallogr 60, 2126-2132
44. Davis, I. W., Murray, L. W., Richardson, J. S., and Richardson, D. C. (2004). MOLPROBITY: structure validation and all-atom contact analysis for nucleic acids and their complexes. Nucleic Acids Res 32, W615-619
45. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998). Crystallography & NMR system: A new software suite for macromolecular structure determination. Acta Crystallogr D Biol Crystallogr 54, 905-921
46. Potterton, L., McNicholas, S., Krissinel, E., Gruber, J., Cowtan, K., Emsley, P., Murshudov, G. N., Cohen, S., Perrakis, A., and Noble, M. (2004). Developments in the CCP4 molecular-graphics project. Acta Crystallogr D Biol Crystallogr 60, 2288-2294
47. Mize, G. J., Wang, W., and Takayama, T. K. (2008). Prostate-specific kallikreins-2 and -4 enhance the proliferation of DU-145 prostate cancer cells through protease-activated receptors-1 and -2. Mol Cancer Res 6, 1043-1051
48. Huck, C. W., Pezzei, V., Schmitz, T., Bonn, G. K., and Bernkop-Schnürch, A. (2006). Oral peptide delivery: Are there remarkable effects on drugs through sulfhydryl conjugation? Journal of Drug Targeting 14, 117-125
49. Chambers, A., and Matrisian, L. (1997). Changing views of the role of matrix metalloproteinases in metastasis. Journal of the National Cancer Institute 89, 1260-1270
50. Hidalgo, M., and Eckhardt, S. G. (2001). Development of Matrix Metalloproteinase Inhibitors in Cancer Therapy. Journal of the National Cancer Institute 93, 178-193.
51. Overall, C. M., and Lopez-Otin, C. (2002). Strategies for MMP inhibition in cancer: innovations for the post-trial era. Nature Reviews Cancer 2, 657-672

1. A compound or salt thereof according to Formula I: 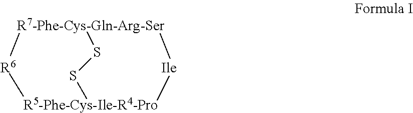
wherein
R⁴, R⁵, R⁶ and R⁷ are each an amino acid residue.

2. (canceled)

3. The compound or salt according to claim 1, wherein and R⁴ is Ala.

4. The compound or salt according to claim 1, wherein R⁶ is Asn.

5. (canceled)

6. The compound according claim 1, wherein the compound is a KLK4 protease inhibitor.

7. A pharmaceutical composition for the treatment of a KLK4 up-regulated cancer in a mammalian subject, the composition comprising the compound according to claim 4 together with a pharmaceutically acceptable carrier or diluent.

8. The pharmaceutical composition of claim 7, wherein the cancer is prostate cancer, ovarian cancer or breast cancer.

9. (canceled)

10. The pharmaceutical composition of claim 7, effective to inhibit or decrease KLK4 protease activity in the subject.

11. The pharmaceutical composition of claim 7, further comprising an anti-cancer agent.

12-13. (canceled)

14. A method for treating a cancer in a mammalian subject, the method comprising the step of administering to the subject a therapeutically effective amount of a pharmaceutical composition comprising the compound according to claim 4.

15. The method according to claim 14, wherein the cancer is prostate cancer, ovarian cancer or breast cancer.

16. A method for treating a KLK4-mediated disease in a mammalian subject, the method comprising the step of administering to the subject a therapeutically effective amount of a pharmaceutical composition comprising the compound according to claim 4.

17. (canceled)

18. The method according to claim 16, wherein the KLK4-mediated disease is a cancer.

19. The method according to claim 18, wherein the cancer is prostate cancer, ovarian cancer or breast cancer.

20. A method for diagnosing a cancer in a mammalian subject, the method comprising the step of contacting a sample from the subject with an effective amount of labelled compound according to claim 4.

21. (canceled)

22. The compound or salt according to claim 1, wherein R6 is Lys.

23. The method according to claim 14, further comprising the step of administering to the subject a therapeutically effective amount of an anti-cancer agent.

24. The method according to claim 23, wherein the cancer is prostate cancer, ovarian cancer or breast cancer.

25. The method according to claim 16, further comprising the step of administering to the subject a therapeutically effective amount of an anti-cancer agent.

26. The method according to claim 20, wherein the cancer is prostate cancer, ovarian cancer or breast cancer.
